(19) United States
(12) Patent Application Publication
(10) Pub. No.: US 2007/0135783 A1
(43) Pub. Date: Jun. 14, 2007

(54) Shield for ostomy patients

(76) Inventor: J. Gordon Scott, Guffey, CO (US)

Correspondence Address: Mr. James G. Scott, 5189 PCR 71, Guffey, CO, 80820, US

(21) Appl. No.: 11/299,555

(22) Filed: Dec. 13, 2005

(51) Int. Cl. A61F 5/44

(52) U.S. Cl. 604/337; 604/345

(57) ABSTRACT

A Shield for protecting an ostomy site from external trauma. A front plate of any material conforming to the curvature of the anatomy proximal to an ostomy site. A pad of any material conforming to the same shape as the front plate installed posterior of front plate in like orientation. An adjustable belt of any material permanently attached to front plate maintains position of shield proximal to ostomy site. A cutout in pad provides a pathway for bodily fluids and waste to transit into the wearers waste receptacle.

### BACKGROUND OF THE INVENTION

1. Field of the invention

The present invention relates to ostomy sites located on or around the lower abdominal quadrant and the devices currently produced to collect liquid and solid waste.

2. Description of the related art.

Human patients having temporary or permanent resection of the colon or ureter in which liquid and or solid waste is routed through an opening in the lower right or left quadrant of the abdomen (stoma). Said waste is collected by means of devices that adhere to the (ostomy) site. Failure of these devices due to common range of motion and or normal external trauma results in loss of containment of said waste, which is common and extremely undesirable.

### SUMARY OF THE INVENTION

The present invention provides external protection for ostomy patients by minimizing external stress to applied ostomy devices.

The present invention distributes the load of normal movement and direct trauma over a large area thus reducing the risk of loss of adhesion.

The present invention allows that ostomy products currently produced to function as intended.

### BRIEF DESCRIPTION OF DRAWINGS

FIG. 1 is a reduced front view of the front plate.

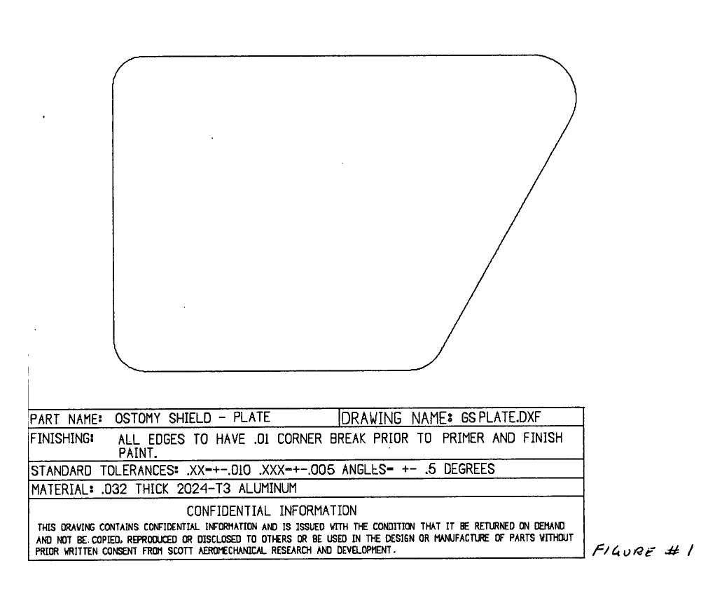

FIG. 2 is a reduced view of the pad that is attached proximally to the anterior aspect of the front plate

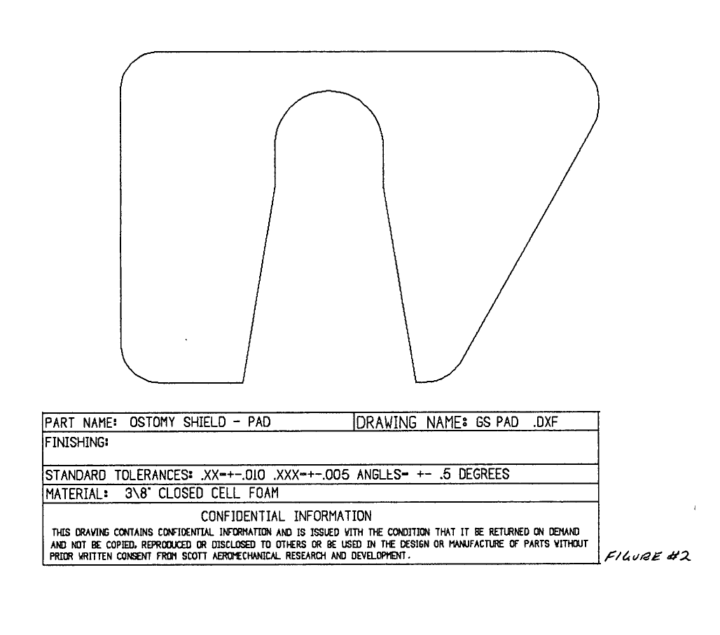

Having illustrated and described the principles of my invention in a preferred embodiment thereof, it should be readily apparent to those skilled in the art that the invention can be modified in arrangement and detail without departing from such principles. I claim all modifications coming within the spirit and scope of the accompanying claims.

I claim:

1. A method of protecting an ostomy site from normal external trauma comprising of: a shield like device that is placed directly over an ostomy site and secured in place by an adjustable belt routed around the patients body and adjusted to a comfortable fit.
